(19) United States
(12) Patent Application Publication
(10) Pub. No.: US 2016/0298120 A1
(43) Pub. Date: Oct. 13, 2016

(54) Interferon Regulatory Factor 1 (IRF1) Decoys and Methods of Use Thereof

(71) Applicant: The Children's Hospital of Philadelphia, Philadelphia, PA (US)

(72) Inventor: Kathleen E. Sullivan, Philadelphia, PA (US)

(21) Appl. No.: 15/187,129

(22) Filed: Jun. 20, 2016

Related U.S. Application Data
(63) Continuation-in-part of application No. PCT/US2014/071359, filed on Dec. 19, 2014.
(60) Provisional application No. 61/918,332, filed on Dec. 19, 2013.

(51) Int. Cl. C12N 15/113; A61K 31/7105; C12N 7/00

(52) U.S. Cl. CPC C12N 15/1136; C12N 7/00; A61K 31/7105; C12N 2310/3231; C12N 2740/16322; C12N 2320/32; C12N 2320/31; C12N 2310/3513

(57) ABSTRACT

Compositions and methods for inhibiting, treating, and/or preventing autoimmune diseases such as lupus are provided.

This application is a continuation-in-part of PCT/US2014/071359, filed on Dec. 19, 2014, which claims priority under claims priority under 35 U.S.C. §119(e) to U.S. Provisional Patent Application No. 61/918,332, filed Dec. 19, 2013. The foregoing applications are incorporated by reference herein.

This invention was made with government support under Grant No. R01 ES017627 awarded by the National Institutes of Health. The Government has certain rights in this invention.

### FIELD OF THE INVENTION

The present invention relates to the field of autoimmune diseases, particularly systemic lupus erythematosus (SLE). Specifically, compositions and methods for treating autoimmune diseases such as lupus are disclosed.

### BACKGROUND OF THE INVENTION

Several publications and patent documents are cited throughout the specification in order to describe the state of the art to which this invention pertains. Each of these citations is incorporated herein by reference as though set forth in full.

Systemic lupus erythematosus (SLE) is a chronic inflammatory disease affecting predominantly young women. Typical manifestations of SLE include a photosensitive rash, glomerulonephritis, arthritis and serositis. In the USA, 1.5 million people are thought to be affected, with annual direct care costs for a person with SLE of $12,643 and annual lost productivity costs of $8659 (Lawrence et al. (2008) Arthritis Rheum., 58:26-35; Zhu et al. (2011) Arthritis Care Res., 63:751-60; Sutcliffe et al. (2001) Rheumatol., 40:37-47). In addition, women with lupus can have compromised fertility and have a rate of temporary disability of approximately 30%, demonstrating the profound impact of this disease. Mortality is thought to be about 2.5 fold higher than the general population with some demographic groups, such as children, having a poor prognosis (Hersh et al. (2010) Arthritis Care Res., 62:1152-9; Cervera et al. (2003) Medicine 82:299-308; Bernatsky et al. (2006) Arthritis Rheum., 54(8):2550-7; MMWR Morb. Mortal Wkly. Rep. (2002) 51:371-4). As a chronic autoimmune disease, the toll can be considerable over the lifetime of a patient. While several new therapeutics have been tested in the last five years, all have a modest effect on disease burden and none have been demonstrated to alter the chronic course of the disease. Accordingly, improved methods of treating lupus are needed.

### SUMMARY OF THE INVENTION

In accordance with the instant invention, IRF1 decoy oligonucleotides are provided. In a particular embodiment, the oligonucleotide comprises a nucleic acid sequence having at least 80% or at least 90% identity with SEQ ID NO: 1 or 2. In a particular embodiment, the oligonucleotide comprises a nucleic acid sequence having at least 80% or at least 90% identity with SEQ ID NO: 12. In a particular embodiment, the oligonucleotide comprises at least one locked nucleic acid. The oligonucleotide may also be conjugated (e.g., via a linker) to at least one cell penetrating peptide. The instant invention also encompasses compositions comprising at least one oligonucleotide of the instant invention and at least one pharmaceutically acceptable carrier.

In accordance with the present invention, methods of inhibiting, treating, and/or preventing an autoimmune disease or disorder such as lupus in a subject are provided. In a particular embodiment, the method comprises administering to the subject at least one IRF1 decoy oligonucleotide. The method may further comprise the administration of at least one other therapeutic agent for the treatment of the autoimmune disease.

### BRIEF DESCRIPTION OF THE DRAWINGS

FIG. 1 provides a schematic of the microbial stimulation or other triggers driving TNF leading to low levels of IFNβ. Together, TNF and IFNβ induce the sustained expression of a variety of mediators and signaling molecules. This process is dependent on IRF1, which acts both as a primer factor by recruiting HATs and also as a direct transcription factor. Activated STAT1, induced by the low level of IFNβ, feeds back to induce additional IRF1.

FIG. 2 provides graphs of flow cytometry performed for specific acetylated lysines in SLE monocytes (left panel) and T cells (middle panel). 20 SLE patients and 15 controls were used. The right panel demonstrates overexpression of mRNA for PCAF/KATB using qRT-PCR in samples from 12 SLE patients and 10 controls. The dark bars denote statistical significance with p<0.05.

FIG. 3 provides a Western blot analysis of MonoMac6 lysates. Cells were treated with indicated stimuli for 24 hours. Co-immunoprecipitations were performed using anti-CBP and anti-PCAF. After immunoprecipitation, the blot was probed with anti-IRF1. This blot demonstrates an association of CBP and PCAF with IRF1. P300 also demonstrated an association but not HAT1 nor ATF2. Equivalent loading was determined by Ponceau staining. N=3.

FIG. 4 provides images of HeLa cells that were treated with low dose TNF, αIFN or γIFN for 2 hours. Immunofluorescence was performed with phalloidin to stain actin and anti-IRF1. Without treatment, IRF1 was perinuclear and after stimulation, it translocated to the nucleus. N=2.

FIG. 5 shows IRF1 binding and H4 acetylation (H4ac) at four genes, as validated by chromatin immunoprecipitation (ChIP). GST was used as a negative control as was globin, for which no detectable IRF1 binding was observed by ChIP-seq. N=3.

FIG. 6A shows a correlation of IRF1 binding and H3K4me³. IRF1 promoter binding sites with increased peak height in SLE patients were selected. H3K4me³ peaks at these sites were amalgamated. Input was used to define the background. H3K4me³ at the nucleosomes directly upstream of IRF1 and immediately downstream of IRF1 were increased. FIG. 6B shows the SLE signal (grey) and the control signal (black) for read depth at the TNFRSF14 locus. The read depths are different for both with a p=4.5e−4 for H3K4me³ and p=1.3e−3 for IRF1.

FIG. 7 shows the effect of decoy molecules. Three decoys were transfected into either K562 or D54MG cells, treated with TNF at 10 ng/ml for 24 hours and then harvested for qRT-PCR using a Taqman 7900HT using gene specific, validated primer/probes (listed on the x-axis). The malaria decoy is the negative control. N=3.

FIG. 8 shows the results of the pull down assays. IRF1CON oligonucleotide was used to pull down IRF1 from untreated and treated MonoMac6 cells. TNF (10 ng/ml) and αIFN (100 U/ml) treatment led to increased binding of IRF1.

FIG. 9 shows the affect of decoy oligonucleotides at certain target genes. The IRF1CON decoy was used to assess the effect on H4 acetylation at three target genes. MonoMac6 cells were transfected with the decoy, treated with 10 ng/ml TNF, and H4 acetylation assessed after 24 hours of stimulation. H4 acetylation was decreased at IRF1 targets in the decoy treated cells. N=2.

FIG. 10 shows the effect of decoy molecules. Two decoys (an IRF1 decoy or LacZ decoy) were transfected into THP1 cells, Jurkat cells, or primary monocytes, the cells were treated with TNF at 10 ng/ml and 100 U/ml α₂IFN for 24 hours, and then the cells were harvested for qRT-PCR using gene specific primer/probes for the indicated genes (CXCL10 (C-X-C motif chemokine ligand 10), CD40 (cluster of differentiation 40), IFIT1 (interferon-induced protein with tetratricopeptide repeats 1), IFIT3 (interferon-induced protein with tetratricopeptide repeats 3), NOD2 (nucleotide-binding oligomerization domain-containing protein 2), B2M (beta 2 microglobulin)). The LacZ decoy is the negative control.

### DETAILED DESCRIPTION OF THE INVENTION

Herein, a novel transcription factor decoy technology is provided to achieve locus specificity by targeting interferon regulatory factor 1 (IRF1)-induced histone modifications. The model system used herein is systemic lupus erythematosus (SLE), a chronic autoimmune disease for which current therapeutics have been only modestly effective. A significant impact of the disease has been demonstrated on the remodeling the epigenome and, therefore, this disease system represents a tractable model for epigenetic intervention (Zhang et al. (2010) Genes Immun., 11:124-33; Sullivan et al. (2007) Clin. Immunol., 123:74-81; Zhang et al. (2010) J. Biomed. Biotechnol., 2010:507475; Lu et al. (2002) Blood 99:4503-8; Lu et al. (2003) J. Immunol., 170:5124-32; Lu et al. (2007) J. Immunol., 179:6352-8; Zhao et al. (2010) J. Autoimmun., 35:58-69; Javierre et al. (2010) Genome Res., 20:170-9). The activation of IRF1 is persistent when both low levels of type I interferons and moderately high levels of TNFα occur. A feed-forward loop leads to persistent activation of both target genes and IRF1 itself (Yarilina et al. (2008) Nat. Immunol., 9:378-87). This combination of cytokines arises in the course of SLE and IRF1 is a significant mediator of inflammation in murine models (Weckerle et al. (2012) Arthritis Rheum., 64:2947-52; Hua et al. (2006) Arthritis Rheum., 54:1906-16; Kirou et al. (2004) Arthritis Rheum., 50:3958-67; Reilly et al. (2006) Eur. J. Immunol., 36:1296-308). Patients with SLE have increased IRF1 binding to chromatin and IRF1 targets have increased histone marks of activation (H4 acetylation, H3K4me³) and gene expression. A study of H4 acetylation at the TNFα locus demonstrated that H4 acetylation correlated with active inflammation in SLE patients, indicating that the histone modifications may be driven by active disease (Sullivan et al. (2007) Clin. Immunol., 123:74-81). High levels of disease activity are known to be associated with elevated serum cytokines (Weckerle et al. (2012) Arthritis Rheum., 64:2947-52; Alvarado-de la Barrera et al. (1998) Scand. J. Immunol., 48:551-6; Aringer et al. (2002) Lupus 11:102-8; Aringer et al. (2003) Arthritis Res. Ther., 5:172-7; Davas et al. (1999) Clin. Rheumatol., 18:17-22; Studnicka-Benke et al. (1996) Brit. J. Rheumatol., 35:1067-74; Yao et al. (2010) Arthritis Res. Ther., 12:S6; Baechler et al. (2003) Proc. Natl. Acad. Sci., 100:2610-5; Bauer et al. (2006) PLoS Med., 3:e491; Bengtsson et al. (2000) Lupus 9:664-71; Dall'era et al. (2005) Ann. Rheum. Dis., 64:1692-7; Kim et al. (1987) Clin. Exp. Immunol., 70:562-9; Ytterberg et al. (1982) Arthritis Rheum., 25:401-6). The sequestering of IRF1 via a decoy molecule as described herein will reset the histone modifications at IRF1-targeted genes and thereby achieve a durably normalized epigenome.

The causes of SLE are not fully known, although a combination of environmental factors and polygenic susceptibility factors are suspected. The disease has four cardinal immunologic features: (1) B cell dysfunction leading to autoantibodies directed at nuclear constituents (Katsumata et al. (1999) Clin. Immunol., 93:75-80; Stauffer et al. (2001) Immunity 15:591-601; Buro et al. (2010) Epigenetics Chromatin 3:16); (2) neutrophil dysfunction associated with disease activity (Molad et al. (1994) Clin. Immunol. Immunopathol., 71:281-6; Ono et al. (1987) J. Virol., 61:2059-62; Ogasawara et al. (1998) Nature 391:700-3); (3) T cell dysfunction, which may enhance autoantibody dysfunction (Maksakova et al. (2008) Cell. Mol. Life Sci., 65:3329-47; Feber et al. (2011) Genome Res., 21:515-24; Choo et al. (2006) Curr. Gene Ther., 6:543-50; Lieberman et al. (2010) J. Biomed. Biotechnol., 2010:740619); and (4) monocyte dysfunction, which may be associated with end-organ inflammation (Feinberg et al. (1983) Nature 301:89-92; Gama-Sosa et al. (1983) Nucleic Acids Res., 11:6883-94; Battistini, A. (2009) J. Interferon Cytokine Res., 29:765-80). Therefore, the disease exhibits not just pleomorphic end organ effects but also pleomorphic immunologic effects.

As stated hereinabove, there is a clearly altered epigenome in SLE patients with a global increase in H4 acetylation. Further, alterations in DNA methylation has also been demonstrated in SLE (Javierre et al. (2010) Genome Res., 20:170-9; Medstrand et al. (1992) J. Gen. Virol., 73:2463-6; Yin et al. (1997) AIDS Res. Hum. Retroviruses 13:507-16; Hohenadl et al. (1999) J. Invest. Dermatol., 113:587-94). These data indicate that the epigenome in SLE is systematically altered. The altered epigenome contributes both to disease phenotype and also to persistence of disease by facilitating pathologic gene expression in immunologically competent cells. The identification of a new strategy to reestablish baseline gene expression is powerful not just for SLE, but as a model for other chronic autoimmune diseases. Indeed, the classic type I interferon gene expression signature, originally identified in SLE, has now been seen in a variety of autoimmune diseases (Salajegheh et al. (2010) Ann. Neurol., 67:53-63; Walsh et al. (2007) Arthritis Rheum., 56:3784-92; Bos et al. (2009) Genes Immun., 10:210-8; van Baarsen et al. (2008) PLoS ONE 3:e1927; Kasperkovitz et al. (2004) Ann. Rheum. Dis., 63:233-9). Therefore, the instant invention has broad applications for the treatment, inhibition, and/or prevention of autoimmune diseases.

As explained herein, an IRF1 decoy is used to alter the histone acetylation pattern in lupus cells. Pioneer transcription factors are critical for chromatin remodeling and lineage commitment (Zaret et al. (2011) Genes Devel., 25:2227-41). A second layer of transcription factors sit poised at genes with potential for upregulation and prime the genes for future expression, largely through the recruitment of histone modifier enzymes (Garber et al. (2012) Molecular Cell. 47:810-22). The third layer represents transcription factors that dynamically bind after a stimulus is given. IRF1 binds to some genes at baseline but is rapidly recruited to inducible promoters/enhancers after stimulation, thereby acting as a hybrid of layer 2 and layer 3. In SLE, chronic low level stimulation by TNF and type I interferons drives IRF1 onto promoters. IRF1 recruits histone acetyltransferases to the chromatin which then acetylate H3 and H4. The increased H3 and H4 acetylation facilitates pathologic gene expression patterns. While current treatment for SLE can diminish active inflammation, persistence is in part due to the altered chromatin landscape that favors a pathologic pattern of gene expression. Therapeutically manipulating the histone acetylation pattern by removing IRF1 is a novel therapeutic strategy.

Immunomodulation resulting from the use of histone deacetylase (HDAC) inhibitors has been studied in murine models. For example, trichostatin (TSA) or the chemically related compound, SAHA, has been utilized in MRL/lpr mice, which normalized RNA and protein levels for pathologically over-expressed proteins and led to less active renal disease (Reilly et al. (2004) J. Immunol., 173:4171-8; Mishra et al. (2003) J. Clin. Invest., 111:539-52; Mishra et al. (2001) Proc. Natl. Acad. Sci., 98:2628-33). Treatment with FR901228, a newer HDAC inhibitor, led to profound immunosuppression, providing an additional explanation for the effect (Skov et al. (2003) Blood 101:1430-8). Diminished DNMT1 activity and increased H3ac was seen in human SLE T cells, while hypoacetylation of H3 and H4 was also identified (Zhao et al. (2010) J. Autoimmun., 35:58-69; Hu et al. (2008) J. Rheumatol., 35:804-10). A study of PBMCs found increased H3K4me³, consistent with the finding of globally increased H4ac (Dai et al. (2010) Clin. Exp. Rheumatol., 28:158-68). Other studies have focused on DNA methylation, finding a demethylated genome with overexpression of many genes central of the aberrant immune response in SLE and that gene expression was tightly correlated with demethylation (Zhu et al. (2011) Int. J. Dermatol., 50:697-704; Balada et al. (2012) PLoS ONE 7:e45897; Sawalha et al. (2012) J. Autoimmun., 38:J216-22; Jeffries et al. (2011) Epigenetics 6:593-601; Mazari et al. (2007) Proc. Natl. Acad. Sci., 104:6317-22; Quddus et al. (1993) J. Clin. Invest., 92:38-53). An SLE twin study found that twins discordant for disease had highly divergent DNA methylomes, supporting the concept that epigenetics are important in the SLE disease process (Javierre et al. (2010) Genome Res., 20:170-9). These studies demonstrate that the epigenome is altered and a targeted epigenetic therapeutic would be therapeutically beneficial.

Interferon regulatory factor 1 (IRF1) was originally identified as a regulator of the interferon β (IFNβ) gene (Miyamoto et al. (1988) Cell 54:903-13). IRF family members all recognize a similar consensus core sequence of AANNNGAAA (SEQ ID NO: 7; Fujii et al. (1999) The EMBO J., 18:5028-41). IRF1 is induced in response to interferons, TNF, LPS, and retinoids and knockout mice have Th2-skewed responses, indicating a role in inflammation (Taki et al. (1997) Immunity 6:673-9). IRFs are phosphorylated in response to stimulation and dimerize, translocating to the nucleus (Lin et al. (1999) Mol. Cell Biol., 19:2465-74; Watanabe et al. (1991) Nuc. Acids Res., 19:4421-8; Sharf et al. (1997) J. Biol. Chem., 272:9785-92). Examples of the amino acid and nucleotide sequences of IRF1 are provided in GenBank GeneID: 3659 and GenBank Accession Nos.: NM_002198.2 and NP_002189.1.

The combination of type I interferons (such as IFNβ) and TNF drives a sustained autocrine loop dependent on IRF1 and STAT1 (Yarilina et al. (2008) Nat. Immunol., 9:378-87; Pollara et al. (2006) Scand. J. Immunol., 63:151-4). An autocrine loop with STAT1 driving more IRF1 and IRF1 contributing to STAT1 activation was defined although NFκB was also required for the full effect. An important consequence of this feed-forward loop was a sustained expression of chemokines which have been implicated in lupus (Menke et al. (2008) J. Amer. Soc. Nephrol., 19:1177-89; Lit et al. (2006) Ann. Rheum. Dis., 65:209-15; Avihingsanon et al. (2006) Kidney Int., 69:747-53; Flier et al. (2001) J. Pathol., 194:398-405). IRF1 is known from other studies to collaborate with NFκB (Shultz et al. (2009) J. Interferon Cytokine Res., 29:817-24; Garber et al. (2012) Molecular Cell 47:810-22). In rheumatoid arthritis, the synovial lining cells were compared with those from patients with osteoarthritis and overexpression of CCL5, CXCL9, CXCL10, STAT1 and IRF1 was observed at both the mRNA and protein level (Yoshida et al. (2012) Scand. J. Rheumatol., 41:170-9). This is comparable to what was seen in the in vitro study with exposure of macrophages to TNF and type I interferons.

It was determined that 63% of the genes with H4 hyperacetylation had potential binding sites for IRF1 within the upstream region. The finding of potential IRF1 binding sites was significant because of its role in the response to the type I IFNs and inflammatory signals, mediators known to be overexpressed in SLE (Shultz et al. (2009) J. Interferon Cytokine Res., 29:817-24; Eklund et al. (1999) J. Immunol., 163:6095-105; Kano et al. (2008) Nat. Immunol., 9:34-41; Pine et al. (1992) J. Virol., 66:4470-8; Ramsauer et al. (2007) Proc. Natl. Acad. Sci., 104:2849-54). It was also significant because IRF1 associates with p300/CBP and the associated factor PCAF, also known as KAT2B (Dornan et al. (2004) Mol. Cell Biol., 24:10083-98; Ramsauer et al. (2007) Proc. Natl. Acad. Sci., 104:2849-54; Masumi et al. (2001) J. Biol. Chem., 276:20973-80; Merika et al. (1998) Molecular Cell 1:277-87).

IRF1 is also notable from the perspective of the female preponderance of SLE. Prolactin is immune stimulatory and can break tolerance of high-affinity DNA-reactive B cells (Karmali et al. (1974) Lancet 2:106-7; Peeva et al. (2003) J. Clin. Invest., 111:275-83). Hyperprolactinemia has been reported in 15-33% of patients with SLE and bromocriptine, which inhibits secretion of prolactin, has been shown to reduce SLE clinical activity (Orbach et al. (2012) Clin. Rev. Allergy Immunol., 42:189-98; Petri, M. (2008) Lupus 17:412-5; Leanos-Miranda et al. (2006) Rheumatol., 45:97-101; Walker, S. E. (2001) Lupus 10:762-8; Alvarez-Nemegyei et al. (1998) Lupus 7:414-9). Prolactin also activates IRF1, leading to hyperacetylation of H4 (Book McAlexander et al. (2001) FEBS Letters 488:91-4).

In addition to the above, IRF1KO mice bred onto the MRL/lpr background ameliorated the classic MRL/lpr skin disease (Reilly et al. (2006) Eur. J. Immunol., 36:1296-308). The IRF1KO mice also had less TNF and IL-12 expression in the mesangium and T cells exhibited a Th2 skewing, consistent with less inflammation. MRL/lpr mice typically have severe glomerulonephritis, as is often seen in human patients. The IRF1KO bred onto the MRL/lpr mice was associated with decreased autoantibodies, less glomerular immune complex deposition, diminished glomerulonephritis and less proteinuria. The IRF1KO mice also had improved survival. These data all support an IRF1-directed intervention in human lupus.

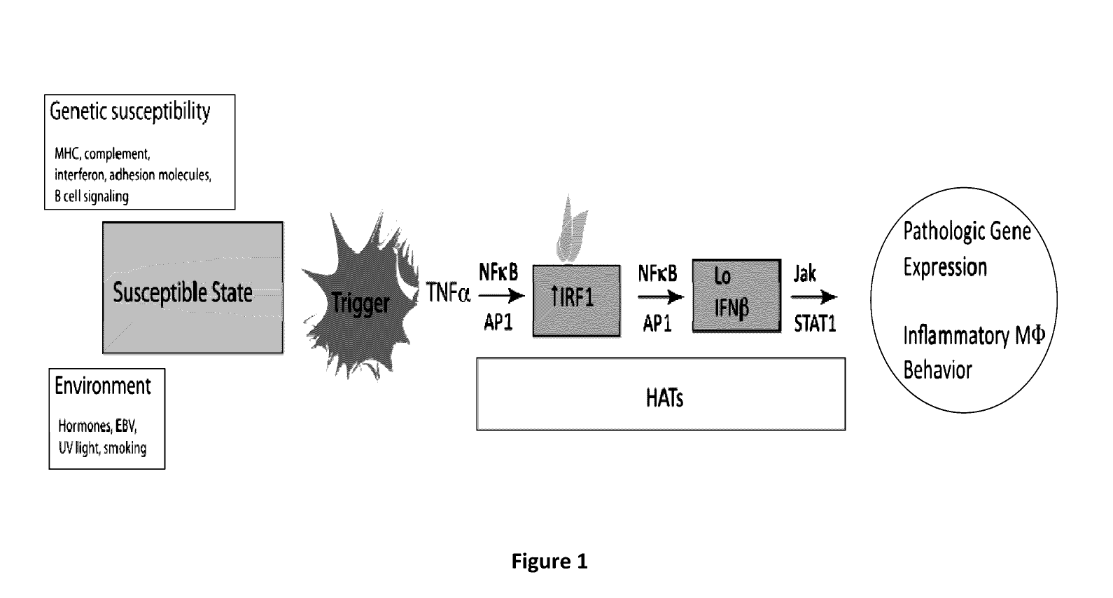

FIG. 1 provides a schematic model for the role of IRF1 in SLE. By using an IRF1 decoy, IRF1 binding on chromatin will be diminished and histone acetylation patterns will be restored to normal specifically at IRF1 target genes.

In accordance with the instant invention, IRF1 decoy oligonucleotides are provided. The oligonucleotides may be used, for example, for the treatment, inhibition, and/or prevention of an autoimmune disease such as lupus. In a particular embodiment, the oligonucleotides (e.g., SEQ ID NO: 3-6, 12) are altered at the variable bases of the IRF1 consensus sequence (e.g., SEQ ID NO: 1 or 2 below). In a particular embodiment, the oligonucleotide sequence may be altered in order to maximize IRF1 binding and specificity. In a particular embodiment, the oligonucleotide comprises a nucleotide sequence having at least 75%, 80%, 85%, 90%, 95%, 97%, 99%, or 100% identity (inclusive of additions, deletions, and/or substitutions) with RAAASNGAAAGTGAAASY (SEQ ID NO: 1; wherein R=A/G, S=G/C, N=any, and Y=C/T), AARAAASNGAAAGTGAAASYC (SEQ ID NO: 2), AAGAAAGAGAAAGTGAAAGTC (SEQ ID NO: 3), AAGAAAGAGAAAGAGAAAGTC (SEQ ID NO: 4), AAGAAAGAGTAAGTGAAAGTC (SEQ ID NO: 5), or AAGAAAGAGAATGTGAAAGTC (SEQ ID NO: 6). In a particular embodiment, the oligonucleotide comprises any one of SEQ ID NOs: 1-6, particularly SEQ ID NO: 1 or 2. The oligonucleotides of the instant invention may comprise any one of SEQ ID NOs: 1-6 at either the 3′ or 5′ end of the oligonucleotide or within the middle of the oligonucleotide.

In a particular embodiment, the oligonucleotide comprises a nucleotide sequence having at least 75%, 80%, 85%, 90%, 95%, 97%, 99%, or 100% identity (inclusive of additions, deletions, and/or substitutions) with GGAAGCGAAAATGAAATTGACT (SEQ ID NO: 12). In a particular embodiment, the oligonucleotide comprises SEQ ID NO: 12. The oligonucleotides of the instant invention may comprise SEQ ID NO: 12 at either the 3′ or 5′ end of the oligonucleotide or within the middle of the oligonucleotide.

In a particular embodiment, the oligonucleotides of the instant invention have fewer than about 50 nucleotides, fewer than about 40 nucleotides, fewer than about 30 nucleotides, fewer than about 25 nucleotides, fewer than about 22 nucleotides, or about 21 nucleotides. In a particular embodiment, the oligonucleotides of the instant invention have more than about 15 nucleotides, more than about 17 nucleotides, more than about 20 nucleotides, or about 21 nucleotides. In a particular embodiment, the oligonucleotide is about 15 to about 50 nucleotides in length, more typically from about 15 to about 30 nucleotides, about 19 to about 25 nucleotides, or about 19 to about 22 nucleotides. The oligonucleotides may be single- or double-stranded. In a particular embodiment, the oligonucleotides are double stranded DNA.

The oligonucleotides of the instant invention may comprise modifications to render the oligonucleotide more resistant to nucleases and/or more capable of entering cells (e.g., either alone or in complex with delivery reagents (e.g., lipid-based transfection reagents)). One (e.g., sense) or both strands of the oligonucleotide may be modified. The oligonucleotides may be modified such that they are capable of entering the nucleus. The oligonucleotides of the instant invention may be designed to have high affinity and specificity for IRF1. A balance should be achieved between having the oligonucleotide too short (as this may reduce binding affinity) or too long (as this may lead to “off-target” effects and/or alter other cellular pathways).

The oligonucleotides of the instant invention may comprise at least one nucleotide analog. The nucleotide analogs may be used to increase annealing affinity, specificity, bioavailability in the cell and organism, cellular and/or nuclear transport, stability, and/or resistance to degradation. Nucleotide analogs include, without limitation, nucleotides with phosphate modifications comprising one or more phosphorothioate, phosphorodithioate, phosphodiester, methyl phosphonate, phosphoramidate, methylphosphonate, phosphotriester, phosphoroaridate, morpholino, amidate carbamate, carboxymethyl, acetamidate, polyamide, sulfonate, sulfonamide, sulfamate, formacetal, thioformacetal, and/or alkylsilyl substitutions (see, e.g., Hunziker and Leumann (1995) Nucleic Acid Analogues: Synthesis and Properties, in Modern Synthetic Methods, VCH, 331-417; Mesmaeker et al. (1994) Novel Backbone Replacements for Oligonucleotides, in Carbohydrate Modifications in Antisense Research, ACS, 24-39); nucleotides with modified sugars (see, e.g., U.S. Patent Application Publication No. 2005/0118605) and sugar modifications such as 2′-O-methyl (2′-O-methylnucleotides) and 2′-O-methyloxyethoxy; and nucleotide mimetics such as, without limitation, peptide nucleic acids (PNA), morpholino nucleic acids, cyclohexenyl nucleic acids, anhydrohexitol nucleic acids, glycol nucleic acid, threose nucleic acid, and locked nucleic acids (LNA) (see, e.g., U.S. Patent Application Publication No. 2005/0118605). See also U.S. Pat. Nos. 5,886,165; 6,140,482; 5,693,773; 5,856,462; 5,973,136; 5,929,226; 6,194,598; 6,172,209; 6,175,004; 6,166,197; 6,166,188; 6,160,152; 6,160,109; 6,153,737; 6,147,200; 6,146,829; 6,127,533; and 6,124,445. The oligonucleotides of the instant invention may also be conjugated or linked to at least one cell penetrating peptide. For example, the oligonucleotides may be linked (e.g., covalently attached) to a tat peptide for delivery (Midoux et al. (1999) Bioconjug. Chem., 10:406-11; Asayama et al. (1998) Bioconjug Chem., 9:476-81; Forrest et al. (2002) Mol. Ther., 6:57-66; Chauhan et al. (2007) J. Control Rel., 117:148-62; Ho et al. (2001) Cancer Res., 61:474-7).

In a particular embodiment of the instant invention, the oligonucleotides of the instant invention comprise one or more locked nucleic acids (LNA). The oligonucleotide may comprise all LNAs or may comprise one or more LNAs present throughout the oligonucleotide and/or at the ends of the oligonucleotide (e.g., each termini of the oligonucleotide may comprise 2 or more (e.g., 2-5) LNAs)). In a particular embodiment, the three 5′ bases and the two 3′ bases are LNAs. LNA oligonucleotides, characterized by a methylene linkage between the 2′-oxygen and the 4′-carbon atoms, have high affinity to complementary DNA while improving the stability of the oligonucleotide and the duplex molecule (Jepsen et al. (2004) Oligonucleotides 14:130-46; Kauppinen et al. (2005) Drug Discov. Today Tech., 2:287-290; Orum et al. (2004) Letters Peptide Sci., 10:325-334). They have low toxicity in biological systems and good aqueous stability (Jepsen et al. (2004) Oligonucleotides 14:130-46; Crinelli et al. (2002) Nuc. Acids Res., 30:2435-43; Fluiter et al. (2003) Nuc. Acids Res., 31:953-62; Kurreck et al. (2002) Nuc. Acids Res., 30:1911-8). LNA oligonucleotides have been successfully administered to primates without demonstrable toxicity, making them an important therapeutic tool (Elmen et al. (2008) Nature 452:896-9; Sen et al. (2012) Cancer Discov., 2:694-705; Sen et al. (2009) Cancer Chemother. Pharmacol., 63:983-95). Importantly, a few LNA bases at the ends are sufficient to resist exonucleases without altering the binding site. LNA oligos do not activate CpG recognition pathways due to their structure (Vollmer et al. (2002) Antisense Nuc. Acid Drug Dev., 12:165-75; Vollmer et al. (2004) Oligonucleotides 14:23-31). Short single-stranded LNA oligonucleotides have shown to be taken up very well by cells and have been shown in a non-human primate model to be taken up by most organs with no evidence of toxicity, thereby supporting this strategy (Elmen et al. (2008) Nature 452:896-9; Sen et al. (2012) Cancer Discov., 2:694-705; Sen et al. (2009) Cancer Chemother. Pharmacol., 63:983-95). Therefore, LNA oligonucleotides offer a combination of features compatible with future clinical use.

The instant invention also encompasses compositions comprising at least one oligonucleotide of the instant invention and at least one carrier (e.g., at least one pharmaceutically acceptable carrier). The compositions of the instant invention may further comprise at least one other therapeutic for the treatment of the autoimmune disease (inclusive of combination therapies). For example, the other therapeutic may be an immunosuppressant (e.g., cyclophosphamide, azathioprine, mycophenolate, leflunomide, methotrexate, and belimumab), antimalarial drug (e.g., hydroxychloroquine), corticosteroid (e.g., prednisone), CD20 antibodies (e.g., rituximab), CTLA-4 and Fc domain fusion proteins (e.g., abatacept), and/or nonsteroidal anti-inflammatory drug (NSAID). In another embodiment, the other therapeutic agent may be contained in a separate composition comprising at least one carrier (e.g., at least one pharmaceutically acceptable carrier). The instant invention also encompasses kits comprising at least one composition comprising an oligonucleotide of the instant invention and, optionally, at least one composition comprising the other therapeutic agent.

The instant invention also encompasses the in vitro delivery of the oligonucleotides of the instant invention to cells and/or tissues. The method comprises contacting said cell or tissue with at least one oligonucleotide of the instant invention. The oligonucleotides of the instant invention may be delivered directly to the cell or tissue (e.g., as part of a composition) or the oligonucleotides may also be delivered in a delivery vehicle (as explained herein).

In accordance with another aspect of the instant invention, methods of treating, inhibiting, and/or preventing an autoimmune disease and/or a disease or disorder characterized by aberrant IRF1 activity (e.g., over-expression, improperly regulated activity, increased activity compared to normal/healthy subject, etc.) are provided. The method comprises administering at least one oligonucleotide of the instant invention to a subject. In a particular embodiment, the autoimmune disease is lupus, dermatomyositis, rheumatoid arthritis, or inflammatory bowel disease, particularly lupus. The oligonucleotides of the instant invention may be administered directly to the subject (e.g., as part of a pharmaceutical composition). The oligonucleotides may also be administered in a delivery vehicle. In a particular embodiment, the oligonucleotide is administered to a cell or organism via an expression vector. An expression vector allows for the expression of the sequences encoded within the nucleic acid construct and/or for the intracellular delivery of the construct. For example, the oligonucleotide can be expressed from a vector such as a plasmid or a viral vector. Examples of viral vectors include, without limitation, adenoviral, retroviral, lentiviral, adeno-associated virus, herpesviral, and vaccinia virus. Expression of such short oligonucleotides from a plasmid or virus has become routine and can be easily adapted to express the oligonucleotides of the instant invention. Other delivery vehicles include, without limitation lipid based vehicles (e.g., liposomes) and biodegradable polymer microspheres. As shown in the examples, nucleofection may be used to deliver the oligonucleotides. Transfection (e.g., lipid mediated), endosomal delivery, and peptide-mediated delivery may also be used. For example, poly-L-lysine or Lipofectamine™ may be used to deliver the oligonucleotides to cells.

The methods of the instant invention may further comprise administering at least one other therapeutic agent for the treatment, inhibition, and/or prevention of the autoimmune disease. Other therapeutic agents are described hereinabove. The other therapeutic agents may be administered sequentially and/or simultaneously with the oligonucleotides of the instant invention.

The compositions of the present invention can be administered by any suitable route, for example, by injection (e.g., for local (direct) or systemic administration), orally, pulmonary, topical, nasally or other modes of administration. The composition may be administered by any suitable means, including parenteral, intramuscular, intravenous, intraarterial, intraperitoneal, subcutaneous, topical, inhalatory, transdermal, intrapulmonary, intraareterial, intrarectal, intramuscular, and intranasal administration. In a particular embodiment, the composition is administered intravenously. In general, the pharmaceutically acceptable carrier of the composition is selected from the group of diluents, preservatives, solubilizers, emulsifiers, adjuvants and/or carriers. The compositions can include diluents of various buffer content (e.g., Tris HCl, acetate, phosphate), pH and ionic strength; and additives such as detergents and solubilizing agents (e.g., Tween® 80, Polysorbate 80), anti oxidants (e.g., ascorbic acid, sodium metabisulfite), preservatives (e.g., Thimersol, benzyl alcohol) and bulking substances (e.g., lactose, mannitol). The compositions can also be incorporated into particulate preparations of polymeric compounds such as polyesters, polyamino acids, hydrogels, polylactide/glycolide copolymers, ethyl enevinylacetate copolymers, polylactic acid, polyglycolic acid, etc., or into liposomes. Such compositions may influence the physical state, stability, rate of in vivo release, and rate of in vivo clearance of components of a pharmaceutical composition of the present invention (see, e.g., Remington: The Science and Practice of Pharmacy, Philadelphia, Pa. Lippincott Williams & Wilkins). The pharmaceutical composition of the present invention can be prepared, for example, in liquid form, or can be in dried powder form (e.g., lyophilized for later reconstitution).

As used herein, “pharmaceutically acceptable carrier” includes any and all solvents, dispersion media and the like which may be appropriate for the desired route of administration of the pharmaceutical preparation, as exemplified in the preceding paragraph. The use of such media for pharmaceutically active substances is known in the art. Except insofar as any conventional media or agent is incompatible with the molecules to be administered, its use in the pharmaceutical preparation is contemplated.

The dose and dosage regimen of the molecule of the invention that is suitable for administration to a particular patient may be determined by a physician considering the patient's age, sex, weight, general medical condition, and the specific condition and severity thereof for which the inhibitor is being administered. The physician may also consider the route of administration, the pharmaceutical carrier, and the molecule's biological activity.

Selection of a suitable pharmaceutical preparation depends upon the method of administration chosen. For example, the molecules of the invention may be administered by direct injection into renal tissue or into the area surrounding the kidneys. In this instance, a pharmaceutical preparation comprises the molecules dispersed in a medium that is compatible with the renal tissue.

Molecules of the instant invention may also be administered parenterally by intravenous injection into the blood stream, or by subcutaneous, intramuscular, intrathecal, or intraperitoneal injection. Pharmaceutical preparations for parenteral injection are known in the art. If parenteral injection is selected as a method for administering the molecules, steps should be taken to ensure that sufficient amounts of the molecules reach their target cells to exert a biological effect. The lipophilicity of the molecules, or the pharmaceutical preparation in which they are delivered, may have to be increased so that the molecules can arrive at their target locations. Methods for increasing the lipophilicity of a molecule are known in the art.

Pharmaceutical compositions containing a compound of the present invention as the active ingredient in intimate admixture with a pharmaceutical carrier can be prepared according to conventional pharmaceutical compounding techniques. The carrier may take a wide variety of forms depending on the form of preparation desired for administration, e.g., intravenous, oral, topical, or parenteral. In preparing the molecule in oral dosage form, any of the usual pharmaceutical media may be employed, such as, for example, water, glycols, oils, alcohols, flavoring agents, preservatives, coloring agents and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Because of their ease in administration, tablets and capsules represent the most advantageous oral dosage unit form in which case solid pharmaceutical carriers are obviously employed. If desired, tablets may be sugar-coated or enteric-coated by standard techniques. For parenterals, the carrier will usually comprise sterile water, though other ingredients, for example, to aid solubility or for preservative purposes, may be included. Injectable suspensions may also be prepared, in which case appropriate liquid carriers, suspending agents and the like may be employed.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art. The appropriate dosage unit for the administration of the molecules of the instant invention may be determined by evaluating the toxicity of the molecules in animal models. Various concentrations of pharmaceutical preparations may be administered to mice with an autoimmune disease (e.g., lupus), and the minimal and maximal dosages may be determined based on the results of significant reduction of the disease and side effects as a result of the treatment. Appropriate dosage unit may also be determined by assessing the efficacy of the treatment in combination with other standard therapies. The dosage units of the molecules may be determined individually or in combination with each therapy according to greater reduction of symptoms and disease.

The pharmaceutical preparation comprising the molecules of the instant invention may be administered at appropriate intervals, for example, at least twice a day or more until the pathological symptoms are reduced or alleviated, after which the dosage may be reduced to a maintenance level. The appropriate interval in a particular case would normally depend on the condition of the patient.

### DEFINITIONS

The following definitions are provided to facilitate an understanding of the present invention:

The singular forms “a,” “an,” and “the” include plural referents unless the context clearly dictates otherwise.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween® 80, Polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in Remington: The Science and Practice of Pharmacy, (Lippincott, Williams and Wilkins); Liberman, et al., Eds., Pharmaceutical Dosage Forms, Marcel Decker, New York, N.Y.; and Rowe, et al., Eds., Handbook of Pharmaceutical Excipients, Pharmaceutical Pr.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., lupus) resulting in a decrease in the probability that the subject will develop the condition.

A “therapeutically effective amount” of a compound or a pharmaceutical composition refers to an amount effective to prevent, inhibit, or treat a particular disorder or disease and/or the symptoms thereof.

As used herein, the term “subject” refers to an animal, particularly a mammal, particularly a human.

The term “isolated” refers to the separation of a compound from other components present during its production. “Isolated” is not meant to exclude artificial or synthetic mixtures with other compounds or materials, or the presence of impurities that do not substantially interfere with the fundamental activity, and that may be present, for example, due to incomplete purification, or the addition of stabilizers.

The terms “linker”, “linker domain”, and “linkage” refer to a chemical moiety comprising a covalent bond or a chain of atoms that covalently attaches at least two compounds, for example, an oligonucleotide to another compound such as cell penetrating peptide. The linker can be linked to any synthetically feasible position of the compounds, but preferably in such a manner as to avoid blocking the compounds desired activity. The linker may be biodegradable or non-degradable under physiological environments or conditions.

As used herein, the term “biodegradable” or “biodegradation” is defined as the conversion of materials into less complex intermediates or end products by solubilization hydrolysis under physiological conditions, or by the action of biologically formed entities which can be enzymes or other products of the organism. The term “non-degradable” refers to a chemical structure that cannot be cleaved under physiological condition, even with any external intervention. The term “degradable” refers to the ability of a chemical structure to be cleaved via physical (such as ultrasonication), chemical (such as pH of less than 6 or more than 8) or biological (enzymatic) means.

The term “oligonucleotide,” as used herein, includes a nucleic acid molecules comprised of two or more ribo- and/or deoxyribonucleotides, preferably more than three. The exact size of the oligonucleotide will depend on various factors and on the particular application and use of the oligonucleotide.

As used herein, a “cell penetrating peptide” refers to a peptide having the ability to transduce another compound (e.g., a nucleic acid molecule) into a cell in vitro and/or in vivo. Examples of cell-penetrating peptides include, without limitation, antennapedia, penetratin, TAT, transportan, short amphipathic peptides (e.g., from the Pep-family (e.g., Pep-1) and MPG-family), S4(13)-PV, polyarginine., and polylysine.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

A “vector” is a genetic element, such as a plasmid, cosmid, bacmid, phage or virus, to which another genetic sequence or element (either DNA or RNA) may be attached. The vector may be a replicon so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a nucleic acid or a polypeptide coding sequence in a host cell or organism. An “expression vector” is a vector which facilitates the expression of a nucleic acid or a polypeptide coding sequence in a host cell or organism.

As used herein, the term “autoimmune disease” refers to the presence of an autoimmune response (an immune response directed against an auto- or self-antigen) in a subject. Autoimmune diseases include diseases caused by a breakdown of self-tolerance such that the adaptive immune system responds to self antigens and mediates cell and tissue damage. In a particular embodiment, autoimmune diseases are characterized as being a result of, at least in part, a humoral and/or cell-mediated immune response. Examples of autoimmune disease include, without limitation, rheumatoid arthritis, type 1 diabetes, systemic lupus erythematosus (lupus or SLE), myasthenia gravis, multiple sclerosis, systemic sclerosis, dermatomyositis, polymyositis, psoriasis, spondylitis, Sjogren's syndrome, Graves disease, inflammatory bowel disease, and Crohn's disease.

The terms “immunosuppressant,” as used herein, includes compounds or compositions which suppress immune responses or the symptoms associated therewith. Immunosuppressant include, without limitation, purine analogs (e.g., azathioprine), methotrexate, cyclosporine, leflunomide, mycophenolate, steroids (e.g., glucocorticoid, corticosteroid), prednisone, non-steroidal anti-inflammatory drug (NSAID), chloroquine, hydroxycloroquine, chlorambucil, CD20 antagonist (e.g., rituximab), abatacept, TNF antagonist (e.g., infliximab), macrolides (e.g., pimecrolimus, tacrolimus (FK506), and sirolimus), dehydroepiandrosterone, lenalidomide, CD40 antagonist (e.g., anti-CD40L antibodies), abetimus sodium, BLys antagonists (e.g., belimumab), dactinomycin, bucillamine, penicillamine, leflunomide, mercaptopurine, pyrimidine analogs (e.g., cytosine arabinoside), mizoribine, alkylating agents (e.g., nitrogen mustard, phenylalanine mustard, buslfan, and cyclophosphamide), folic acid antagonsists (e.g., aminopterin and methotrexate), antibiotics (e.g., rapamycin, actinomycin D, mitomycin C, puramycin, and chloramphenicol), antilymphocyte globulin (ALG), antibodies (e.g., anti-CD3 (OKT3), anti-CD4 (OKT4), anti-CD5, anti-CD7, anti-IL-2 receptor (e.g., daclizumab and basiliximab), anti-alpha/beta TCR, anti-ICAM-1, muromonab-CD3, anti-IL-12, alemtuzumab and antibodies to immunotoxins), 1-methyltryptophan, and derivatives and analogs thereof.

The following example is provided to illustrate various embodiments of the present invention. The example is illustrative and is not intended to limit the invention in any way.

### Example

Increased H4ac was originally found at the TNFα promoter in monocytes in SLE compared to controls (Sullivan et al. (2007) Clin. Immunol., 123:74-81). Since, global changes in H4ac in SLE monocytes have been demonstrated. Monocyte behavior is impacted by changes to the epigenome and genes characterized by altered potential for expression after cytokine treatment have durable changes to H4ac with a common theme being regulation by MAP kinases. The association of durably altered gene expression with H4 acetylation in monocytes indicates that the altered epigenome molds cell behavior. MAP kinases play a role in the recruitment of RNA pol II and H4ac, supporting a mechanistic connection. The data indicate that interferon both stimulate the expression of the genes that comprise the type I interferon signature and also durably alter the transcriptional set point of the genes via changes to the epigenome. Significantly, 113 of 179 genes with increased H4ac in SLE had potential IRF1 binding sites (p=10⁻²⁵). An RNA-seq study was performed which found enrichment of potential IRF1 binding sites upstream of overexpressed genes with p=1.4e−3. This is comparable to the ChIP-chip study which found enrichment of potential IRF1 binding sites both in the genes with increased H4ac in SLE and increased expression in SLE, with a high concordance between the two gene sets (p=0.0002).

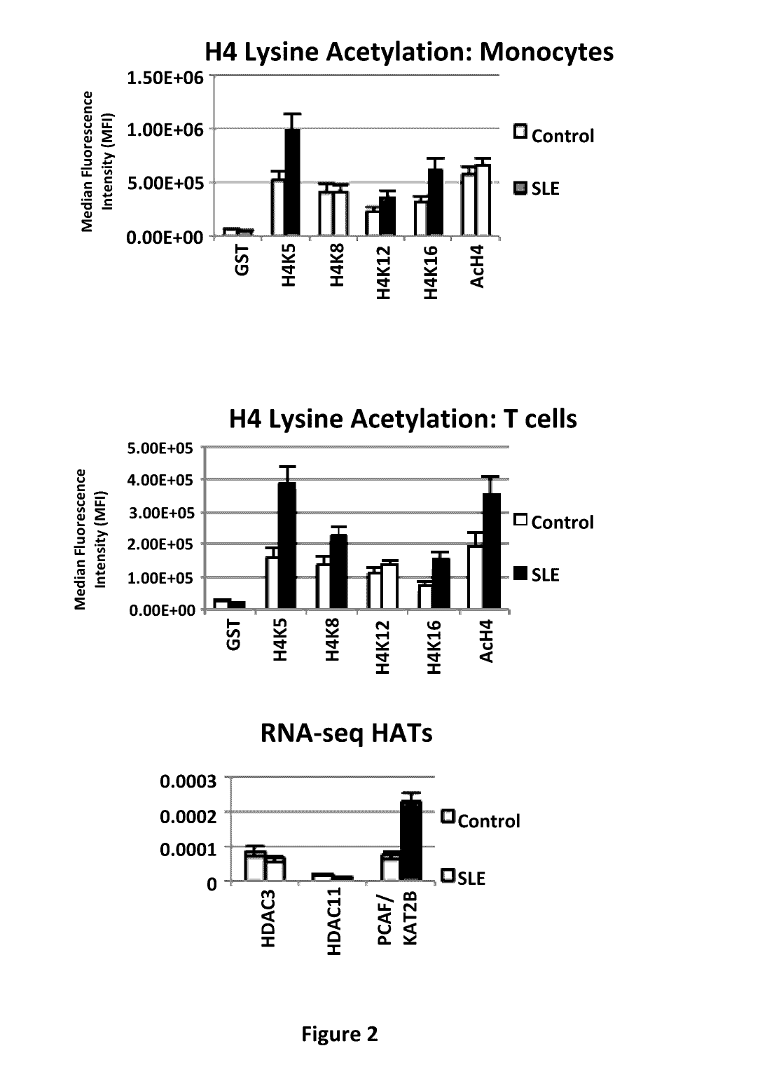

At the time of the ChIP-chip, the best available antibody was directed at pan-acetylated H4. To identify potential histone acetyltransferases (HATs), the lysine moieties that were globally hyperacetylated in SLE were defined. Using a new cohort of SLE patients with low disease activity, flow cytometry was performed looking at T cells and monocytes (FIG. 2). In both T cells and monocytes, H4K5ac and H4K16ac were increased in SLE patients compared to controls. There were differences, however, with H4K8ac increased in T cells and H4K12ac increased in monocytes. Using regression analysis, a strong correlation of global H4K5ac, H4K12ac, and H4K16ac in monocytes and H4K12ac with H4K16ac in T cells was observed. The only modification for which monocytes and T cells were correlated was H4K12ac. These studies did not examine specific genes, but confirm the global hyperacetylation identified in the ChIP-chip study. PCAF/KAT2B has been identified as a HAT associated with IRF1 and preferentially acetylates H4K8 and H4K12, providing some redundancy with GCN5 (Jin et al. (2011) EMBO J., 30:249-62; Nagy et al. (2010) Cell. Mol. Life Sci., 67:611-28; Guelman et al. (2009) Mol. Cell Biol., 29:1176-88; Kikuchi et al. (2005) Gene 347:83-97). As seen in FIG. 2, PCAF/KAT2B is overexpressed in SLE monocytes. Nevertheless, it is clear that additional pathways contribute to the altered H4 acetylation identified in SLE (Nagy et al. (2007) Oncogene 26:5341-57; Maurice et al. (2008) Neuropsychopharmacol., 33:1584-602).

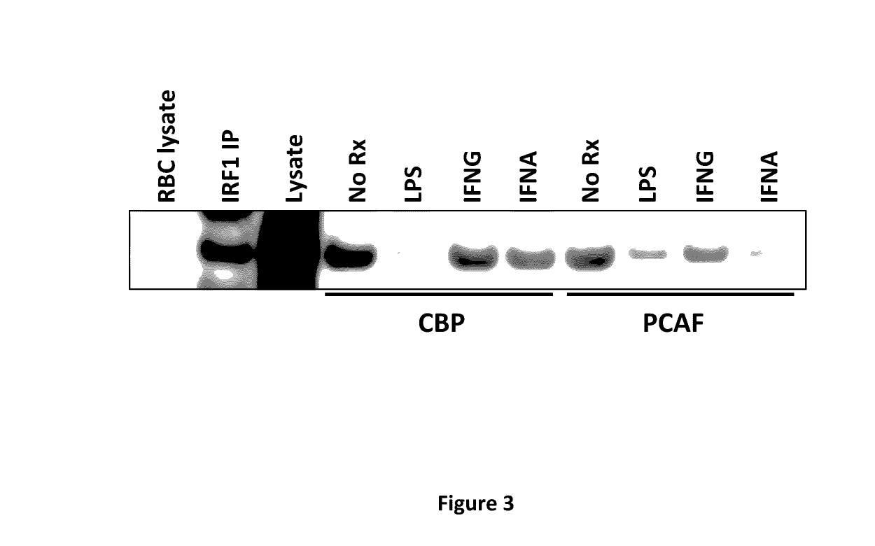

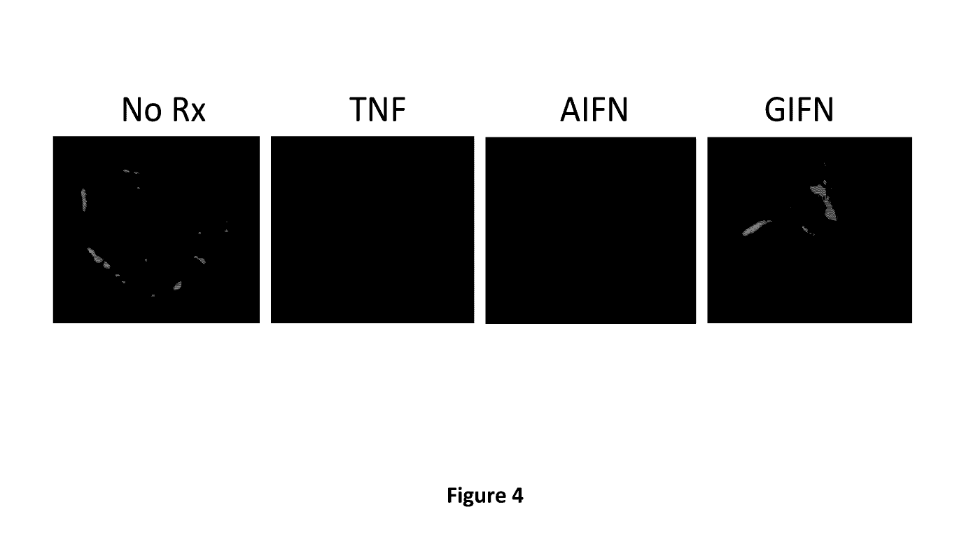

To identify HATs associated with IRF1, co-immunoprecipitations were performed (FIG. 3). An association of PCAF, CBP and p300 (but not ATF2 or HAT1) with IRF1 using co-immunoprecipitation was demonstrated. Little variation was observed across stimuli. Using co-immunoprecipitation, the phosphorylation of IRF1 was also observed. Increased phosphorylation was observed in response to 10 ng/ml of TNF, 500 U/ml of αIFN and 500 U/ml of γIFN. These same stimuli were used and translocation to the nucleus was observed at 2 hours (FIG. 4).

To better understand the role of IRF1, a ChIP-seq was performed to directly identify binding sites. All IRFs were originally thought to bind the same consensus sequence, however, a superior IRF1 binding site was defined using ChIP-seq and recent ChIP-seq studies of IRF8 and IRF5 found binding motifs different than the consensus (Shin et al. (2011) PLoS ONE 6:e27384; Wang et al. (2013) Ann. Rheum. Dis., 72:96-103). The 18 bp consensus derived for IRF1 was superior to the previous 12 bp consensus for the correct identification of genes involved in interferon responses (Shi et al. (2011) Gene 487:21-8). This consensus was used to design the IRF1 decoy (Table 1).

TABLE 1
IRF1 decoy oligonucleotides: The three 5′ bases
and the two 3′ bases have a LNA modification
(italics). The reverse complement is unmodified
in each case. SEQ ID NOs are provided in
parentheses.

| | |
|---|---|
| ChIP-seq consensus | RAAASNGAAAGTGAAASY (1) |
| IRF1 CON | AAGAAAGAGAAAGTGAAAGTC (3) |
| IRF1 IFNG promoter | GAGAAGTGAAAGT (8) |
| IRF1 IFNA1 promoter | CGAAATGGAAAGTA (9) |
| IRF1 Consensus 14A | AAGAAAGAGAAAGAGAAAGTC (4) |
| IRF1 Consensus 10T | AAGAAAGAGTAAGTGAAAGTC (5) |
| IRF1 Consensus 12T | AAGAAAGAGAATGTGAAAGTC (6) |
| Scramble | AAAAAATTCGGGAAAAGCGAA (10) |
| Malaria | AAATATTTTAAAAACATCCTGG (11) |

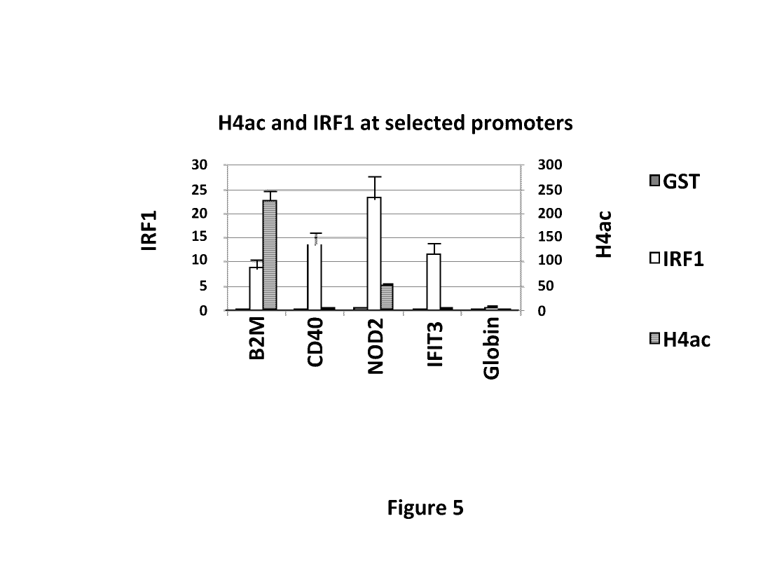

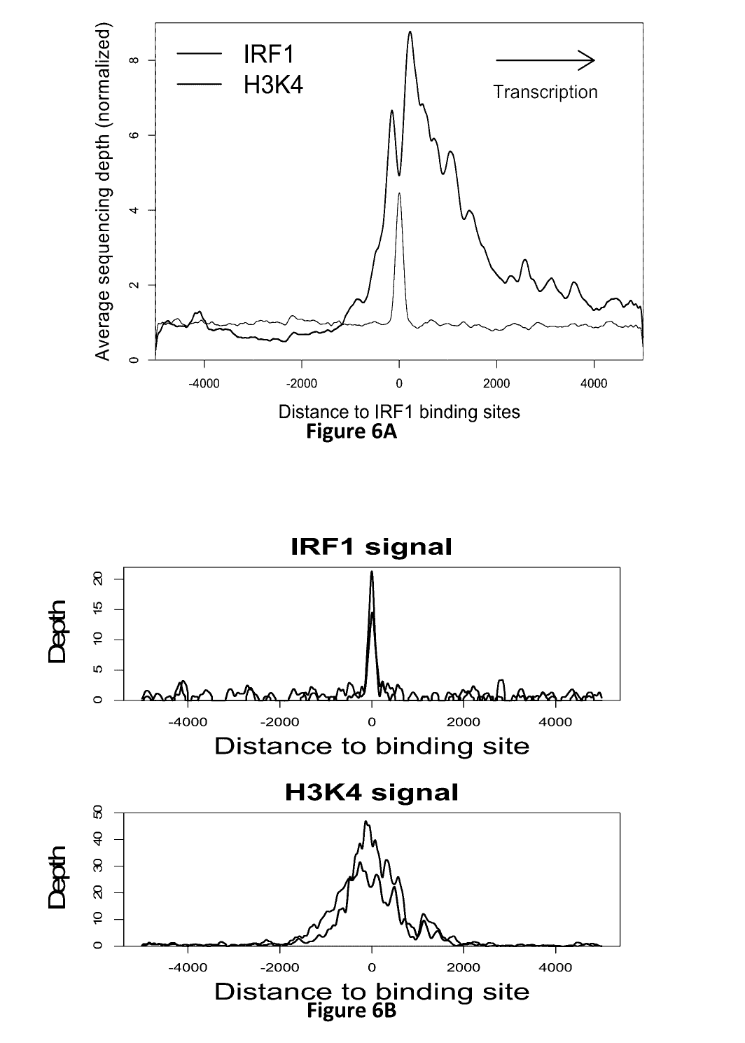

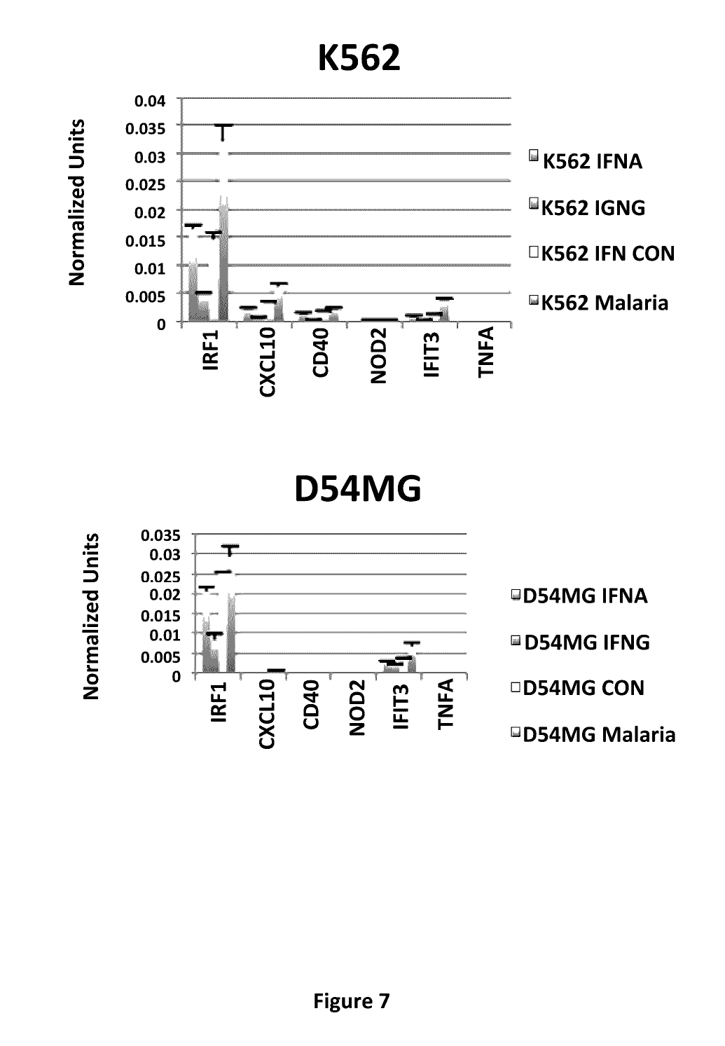

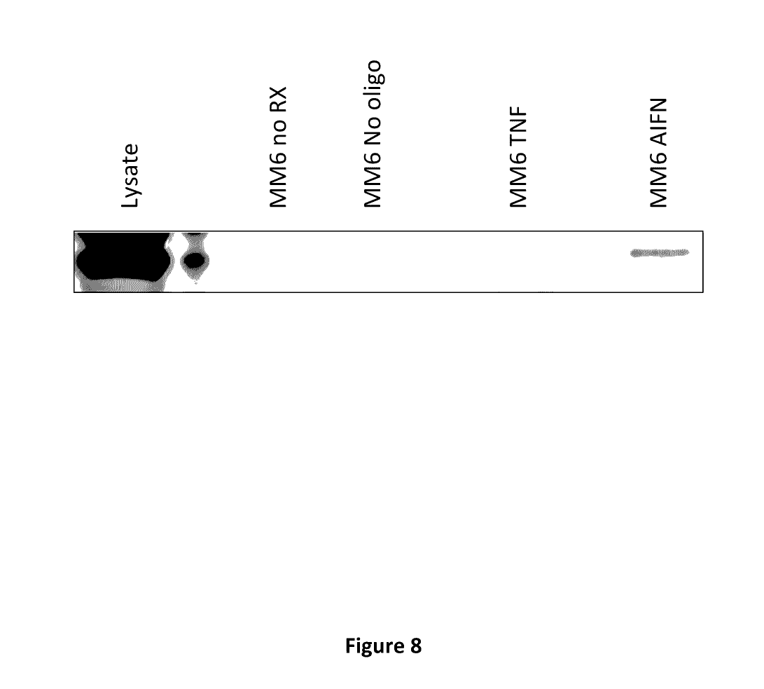

IRF1 binding sites increased in SLE patients were also directly identified. IRF1 ChIP-seq results were analyzed from monocytes from 9 patients and 8 controls. When IRF1 peaks with differential binding and a p<0.05 were analyzed, 78 peaks were identified and 74/78 exhibited increased binding in SLE. The specificity of the ChIP-seq peaks was validated using a standard ChIP assay on primary monocytes and peaks at the focus set of B2M, CD40, NOD2, and IFIT3 were found (FIG. 5). To better identify correlates of IRF1 binding, genome-wide H3K4me³ in the same 9 patients and 8 controls were examined. When IRF1 peaks were identified in the promoter, the IRF1 peaks were uniformly embedded in a peak of H3K4me³ and a trough of H3K27me³ (FIG. 6). There was a very strong association overall between H3K4me³ and IRF1. Increased H3K4me³ was significantly associated with increased IRF1 in SLE patients (p=1.4 e−19). To illustrate the effect at a single gene, the IRF1 binding for SLE patients and controls is shown in FIG. 6 for TNFRSF14 (HVEM), which has been implicated in atherogenesis (Bobik et al. (2001) Arter. Thromb. Vasc. Biol., 21:1873-5). These studies collectively indicate that IRF1 binding is associated with chromatin marks of activation. Causality was investigated by using several decoy molecules. These were synthesized according to the sequences in Table 1 and transfected using nucleofection. The effect of three decoys and a malaria oligonucleotide which is similarly rich in adenine bases were evaluated. K562 and D54MG cells were treated for 24 hours with 10 ng/ml of TNF and mRNA was quantitated using qRT-PCR (FIG. 7). All three decoys significantly diminished the expression of IRF1, CXCL10, IFIT3 and TNF expression in K562. Three of these were significantly decreased in decoy-treated D54MG cells as well. These studies demonstrate that the IRF1 decoys are effective at targeting gene expression. To ensure that the oligonucleotides acted through binding to IRF1, a pull-down assay using a biotinylated IRF1 decoy (non-LNA) corresponding to the IRF1CON sequence was performed. Streptavidin beads were used to collect the oligonucleotide and any associated proteins (FIG. 8).

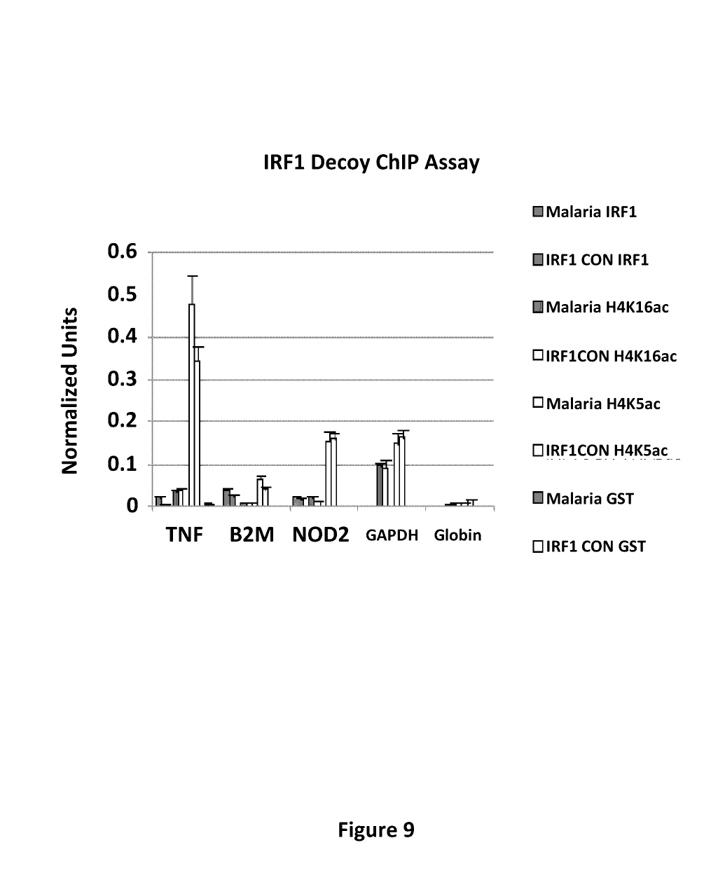

The IRF1CON decoy was also transfected and H4 acetylation was assessed at known targets (FIG. 9). H4K5ac was decreased in IRF1CON-treated MonoMac6 cells at the TNF and B2M genes. H4K16ac was decreased at NOD2. Neither globin (a non-expressed gene) nor GAPDH (an expressed gene but non-IRF1-target) were altered by the decoy.

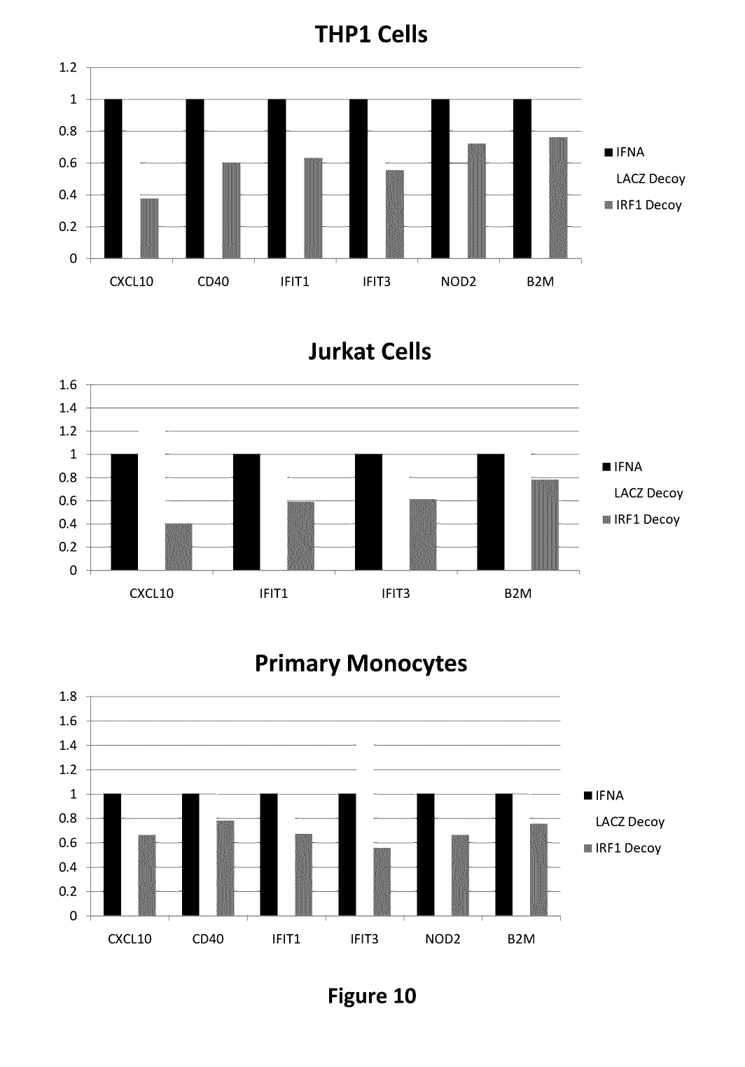

Three additional cell types were assayed with an IRF1 decoy having the sequence GGAAGCGAAAATGAAATTGACT (SEQ ID NO: 12), wherein the three 5′ bases and the two 3′ bases have an LNA modification. THP1 cells (human monocytes), Jurkat cells (human T lymphocytes), and primary monocytes were transfected by nucleofection and treated for 24 hours with 10 ng/ml of TNF and 100 U/ml α₂IFN. mRNA was then quantitated using qRT-PCR (FIG. 10). Compared to a control decoy (LacZ decoy), the IRF decoy significantly diminished the expression of various target genes in all three cell types. These studies demonstrate that the IRF1 decoys are effective at targeting gene expression.

Collectively, these studies demonstrate the use of an IRF1-directed approach to target biologically relevant genes in chronic autoimmune diseases.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. An oligonucleotide comprising a nucleic acid sequence having at least 90% identity with SEQ ID NO: 1, 2, or 12.

2. The oligonucleotide of claim 1, comprising SEQ ID NO: 1, 2, or 12.

3. The oligonucleotide of claim 1, comprising SEQ ID NO: 3, SEQ ID NO: 4, SEQ ID NO: 5, or SEQ ID NO: 6.

4. The oligonucleotide of claim 1, having at least 95% identity SEQ ID NO: 12.

5. The oligonucleotide of claim 1, comprising SEQ ID NO: 12.

6. The oligonucleotide of claim 1, wherein said oligonucleotide comprises at least one locked nucleic acid.

7. The oligonucleotide of claim 6, wherein each termini of the oligonucleotide comprises at least 2 locked nucleic acids.

8. The oligonucleotide of claim 1, wherein said oligonucleotide is less than about 50 nucleotides in length.

9. The oligonucleotide of claim 8, wherein said oligonucleotide is from about 19 to about 25 nucleotides in length.

10. A complex comprising the oligonucleotide of claim 1 linked to at least one cell penetrating peptide.

11. The complex of claim 10, wherein said cell penetrating peptide is a peptide of HIV Tat.

12. A composition comprising at least one oligonucleotide of claim 1 and at least one pharmaceutically acceptable carrier.

13. A method of treating, inhibiting, and/or preventing an autoimmune disease in a subject, said method comprising administering to said subject at least one oligonucleotide of claim 1.

14. The method of claim 13, wherein said autoimmune disease is lupus.

15. The method of claim 13, further comprising administering at least one other therapeutic agent for the treatment of said autoimmune disease.


SEQUENCE LISTING (12 sequences)

SEQ ID NO: 1 - DNA, 18 bp, Artificial Sequence
  Synthetic sequence
raaasngaaagtgaaasy

SEQ ID NO: 2 - DNA, 21 bp, Artificial Sequence
  Synthetic sequence
aaraaasngaaagtgaaasyc

SEQ ID NO: 3 - DNA, 21 bp, Artificial Sequence
  Synthetic sequence
aagaaagagaaagtgaaagtc

SEQ ID NO: 4 - DNA, 21 bp, Artificial Sequence
  Synthetic sequence
aagaaagagaaagagaaagtc

SEQ ID NO: 5 - DNA, 21 bp, Artificial Sequence
  Synthetic sequence
aagaaagagtaagtgaaagtc

SEQ ID NO: 6 - DNA, 21 bp, Artificial Sequence
  Synthetic sequence
aagaaagagaatgtgaaagtc

SEQ ID NO: 7 - DNA, 9 bp, Artificial Sequence
  Synthetic sequence
aannngaaa

SEQ ID NO: 8 - DNA, 13 bp, Artificial Sequence
  Synthetic sequence
gagaagtgaaagt

SEQ ID NO: 9 - DNA, 14 bp, Artificial Sequence
  Synthetic sequence
cgaaatggaaagta

SEQ ID NO: 10 - DNA, 21 bp, Artificial Sequence
  Synthetic sequence
aaaaaattcgggaaaagcgaa

SEQ ID NO: 11 - DNA, 22 bp, Artificial Sequence
  Synthetic sequence
aaatattttaaaaacatcctgg

SEQ ID NO: 12 - DNA, 22 bp, Artificial Sequence
  Synthetic sequence
ggaagcgaaaatgaaattgact